(19) United States
(12) Patent Application Publication
(10) Pub. No.: US 2013/0331690 A1
(43) Pub. Date: Dec. 12, 2013

(54) VASCULAR IMAGING AGENTS

(71) Applicants: Andrew Healey, Moss (NO); Ragnar Bendiksen, Oslo (NO); Anup Sood, Niskayuna, NY (US); Balin Balinov, Oslo (NO)

(72) Inventors: Andrew Healey, Moss (NO); Ragnar Bendiksen, Oslo (NO); Anup Sood, Niskayuna, NY (US); Balin Balinov, Oslo (NO)

(73) Assignee: GE HEALTHCARE AS, OSLO (NO)

(21) Appl. No.: 13/824,466

(22) PCT Filed: Sep. 21, 2011
(86) PCT No.: PCT/EP11/66464

§ 371 Date: Aug. 30, 2013

(30) Foreign Application Priority Data
Sep. 21, 2010 (GB) 1015806.1

(51) Int. Cl. A61K 49/00; A61B 5/00

(52) U.S. Cl. CPC A61K 49/006; A61B 5/0073
USPC 600/425; 600/431

(57) ABSTRACT

The present invention relates to a method of in vivo optical imaging, of the blood vessels and/or blood pool of a mammalian subject, which comprises an optical imaging contrast agent. The optical imaging agents comprise conjugates of far-red or near-infrared dyes with synthetic polyethyleneglycol (PEG) polymers having a molecular weight in the range 15-45 kDa. Also disclosed are methods of treatment monitoring, methods of diagnosis and medical uses involving the contrast agents.

### FIELD OF THE INVENTION

The present invention relates to a method of in vivo optical imaging of blood vessels and/or blood pool, which method comprises an optical imaging contrast agent. The optical imaging agents comprise conjugates of far-red or near-infrared dyes with synthetic polyethyleneglycol (PEG) polymers having a molecular weight in the range 15-45 kDa. Also disclosed are methods of treatment monitoring, methods of diagnosis and medical uses involving the contrast agents.

### BACKGROUND TO THE INVENTION

Wohrle et at [Makromol. Symp., 59, 17-33 (1992)] studied polymer-conjugation to porphyrin photosensitisers as a potential method of improving the uptake in target tissue in vivo for the photodynamic therapy of cancer. The polymers studied were rat serum albumin, synthetic polyethers and polyalcohols. Wohrle et al concluded that the conjugation of a polymer carrier could improve the tumour uptake.

U.S. Pat. No. 5,622,685 discloses that polyether-substituted anti-tumour agents comprising a porphyrin, phthalocyanine or naphthalocyanine exhibit improved properties for both in vivo tumour diagnosis and therapy. The polyether substituents comprise polyethyleneglycol (PEG) whose terminal hydroxyl group is etherified or esterified with C₁₋₁₂ alkyl or C₁₋₁₂ acyl groups respectively. The alkyl group is most preferably a methyl group. U.S. Pat. No. 5,622,685 teaches (column 2) that the total molecular weight of the conjugate is preferably at least 10,000 Da (10 kDa).

U.S. Pat. No. 6,083,485 and counterparts disclose in vivo near-infrared (NIR) optical imaging methods using cyanine dyes having an octanol-water partition coefficient of 2.0 or less. Also disclosed are conjugates of said dyes with “biological detecting units” of molecular weight up to 30 kDa which bind to specific cell populations, or bind selectively to receptors, or accumulate in tissues or tumours. The dyes of U.S. Pat. No. 6,083,485 may also be conjugated to a range of “non-selectively bonding” macromolecules, such as polylysine, dextran, carboxydextran, polyethylene glycol, methoxypolyethylene glycol, polyvinyl alcohol, or a cascade polymer-like structure. The molecular weight of the conjugates is taught to range from 100 Da to over 100,000 Da (0.1 to over 100 kDa). No specific dye-macromolecule conjugates are disclosed.

GB 2,337,523 A (Nycomed Imaging AS) discloses a physiologically tolerable water-soluble light imaging contrast agent having a molecular weight in the range 500 to 500,000 Daltons and containing at least two chromophores having delocalized electron systems that are linked to at least one polymer surfactant moiety having a molecular weight in the range 60 to 100,000. GB 2,337,523 A teaches that the polymer surfactant can be a polyalkylene oxide, polysaccharide or polyvinyl alcohol. GB 2,337,523 A states that small organic chromophores such as indocyanine green (ICG) suffers from rapid blood clearance, and seeks to provide contrast agents which have an extended imaging window, suitable for blood flow studies, perfusion of effusion and the vascularization of sites of interest. GB 2,337,523 A does not, however, teach which specific combinations of surfactant polymer, chromophores and molecular weight solve the stated problem. The Examples of GB 2,337,523 A closely parallel those of U.S. Pat. No. 6,350,431 as described below.

U.S. Pat. No. 6,350,431 (Nycomed Imaging AS) discloses light imaging contrast agents having a molecular weight in the range 500 to 500,000 Da, comprising a polyalkylene oxide (PAO) of molecular weight 60 to 100,000 Da having at least two chromophores (i.e. dye molecules) linked thereto. The polyalkylene oxide (PAO) moiety is taught to have a preferred molecular weight range of 200 to 100,000 Da, more preferably 250 to 50,000 Da, especially preferably 250 to 25,000 Da, most preferably 400 to 15,000 Da. The contrast agents of U.S. Pat. No. 6,350,431 may further comprise a targeting vector. The Examples of U.S. Pat. No. 6,350,431 employ the following PAO polymers
(i) PEG-diamine 3,400 Da molecular weight: Examples 1, 2, 6, 16, 18 and 25;
(ii) PEG-diamine 5,000 Da molecular weight: Examples 3, 4 and 20;
(iii) PEG-diamine 10,000 Da molecular weight: Examples 7, 15, 17 and 26;
(iv) PEG-dithiol 3,400 Da molecular weight: Example 12;
(v) PEG-dithiol 10,000 Da molecular weight: Example 13;
(vi) Poly(oxyethylene-co-oxypropylene-co-oxyethylene) block copolymer of average molecular weight about 14,600: Example 27.

Thus, the Examples of U.S. Pat. No. 6,350,431 are all in the molecular weight range 3.4 to 14.6 kDa. For PEG polymers alone, the molecular weight range exemplified is 3.4 to 10 kDa.

Yuan et al [Cancer Res., 55, 3752-3756 (1995)] studied the vascular permeability of human tumour cells to dye-labelled macromolecules, and concluded that tumor vessels are in general more leaky and less permselective than normal cells. The tumour cell permeability was reported to vary twofold in the macromolecule molecular weight range 25 kDa to 160 kDa.

Dellian et al [Br. J. Cancer, 82(9), 1513-1518 (2000)] studied the effect of molecular charge on the vascular permeability of human tumour cells. They concluded that positively-charged molecules extravasate more quickly into solid tumours compared with neutral or negatively-charged compounds of similar molecular weight.

Licha et at [SPIE Vol 3196 p. 98-102 (1998)] disclose contrast agents for in vivo fluorescence imaging which comprise poly(ethyleneglycol) (PEG) polymers based on methoxypolyethyleneglycol (MPEG). The conjugates thus have a heptamethine cyanine dye conjugated at one terminus of the PEG polymer and a methyl group at the other terminus:

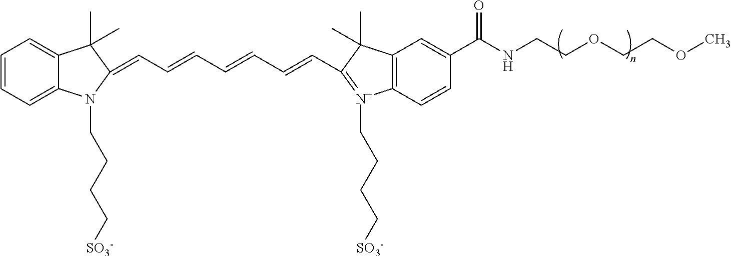

| | | Molecular weight |
|---|---|---|
| Dye conjugate | n | (kDa) |
| NIR96017 | 22-28 | 1.83 |
| NIR96008 | 100-150 | 6.15 |
| NIR96486 | 240-320 | 13.2 |
| NIR96016 | 420-530 | 20.7 |

Also disclosed by Licha was a dye conjugate in which 2 MPEG chains were conjugated to a single cyanine dye (NIR96307, molecular weight ca. 41 kDa):

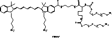

For NIR96307, n was not determined, but the mean molecular weight of the conjugate was said to be 41 kDa. The polymer conjugates of Licha were synthesized from the corresponding MPEG amine, ie. H₂NCH₂[CH₂OCH₂]ₙCH₂OCH₃.

In a related publication [Licha et al, SPIE Vol 3196, p. 103-110 (1998)] describe tumour detection in animals using the above MPEG conjugates. In particular, the interest was in the effect of the molecular weight of the PEG conjugate on: (i) their tolerability; (ii) the pharmacokinetic behaviour; and (iii) the contrast between malignant and normal tissue. They observed that increasing molecular weight prolonged the blood circulation time in vivo. They concluded that increased retention in the tumour environment and improved tumour contrast was observed at later times for dye-MPEG conjugates with a molecular weight above 6 kDa.

Montet et al [Radiology, 242(3), 751-758 (2007)] reported fluorescence molecular tomography (FMT) of angiogenesis using the near-infrared probes AngioSense 680 and AngioSense 750. These were described as high molecular weight (250 kDa) pegylated graft copolymers with an indocyanine-type fluorophore optimized for non-quenching. The agent contains MPEG attached to a polylysine backbone. Montet et al report that the agent exhibited a prolonged blood half-life (more than 5 hours), with no tumour extravasation up to 30 minutes post-administration, but increasing tumour uptake (and hence imaging brightness) with time thereafter.

Sadd et at [J. Control. Rel., 130, 107-114 (2008)] studied the characteristics of 3 different nanocarriers (linear polymer; dendrimers and liposome) on the efficacy of chemotherapy and imaging in vitro and in vivo. The linear polymer studied comprised a targeted PEG polymer of the type:

[LHRH]-[PEG polymer]-Cy5.5
where: LHRH is a synthetic analogue of luteinizing hormone-releasing peptide;
Cy5.5 is a specific cyanine dye.

The PEG polymer used had a molecular weight of about 3 kDa. FIG. 4 (p. 111) of Sadd et al compares the tumour uptake of the above conjugate with the non-targeted analogue, PEG-Cy5.5. Sadd et al concluded that the LHRH targeting polymer conjugate exhibits enhanced accumulation in cancer cells compared to the non-targeted analogue.

Desmetter et al [Surv. Ophthalmol., 45, 15-27 (2000)] reviewed the fluorescence and metabolic properties of the dye indocyanine green (ICG) for use in angiography in vivo—in particular retinal and choroidal vasculature.

WO 2007/000349 discloses the use of indocarbocyanine dyes for optical imaging of rheumatoid arthritis. The method if based on the differential distribution and/or residence of the dye in healthy vs inflamed areas. A preferred dye of WO 2007/000349 is ICG.

Fischer et al [Acad. Radiol., 17, 375-381 (2010)] studied the optical imaging of arthritis using non-specific dyes, and concluded that ICG imaging can detect early inflammatory changes. The optical images were found to correlate well with MRI images.

WO 2010/106169 discloses a method of in vivo optical imaging of the tumour margins of a tumour in an animate subject known to have at least one such tumour, said method comprising:
(i) providing an optical imaging contrast agent suitable for in vivo imaging, said contrast agent comprising a conjugate of a synthetic polyethyleneglycol polymer of molecular weight 15 to 45 kDa, with one or two groups Opt^{R};
(ii) generating an optical image of a region of interest of said subject to which said contrast agent has been administered, said region of interest comprising said tumour and tumour margin;
wherein each Opt^{R} is independently a biocompatible optical reporter group capable of detection either directly or indirectly in an optical imaging procedure using light of wavelength 600-850 nm.

WO 2010/106169 does not disclose blood pool and/or blood vessel imaging using the agents described therein.

### The Present Invention.

The present invention provides an alternative method of in vivo optical imaging of blood vessels and/or blood pool, using an optical imaging contrast agent. The optical imaging agents comprise conjugates of dyes with synthetic polyethyleneglycol (PEG) polymers having a molecular weight in the range 15-45 kDa.

The existing agent indocyanine green (ICG) does have some disadvantages. Thus, ICG has a relatively short blood clearance half-life in vivo of 3.9±0.9 min in healthy subjects [Bax et al, Br. J. Clin. Pharmacol., 10, 353-361 (1980)]. Hence, when the optical data acquisition requires several minutes to perform (e.g. for tomographic imaging), a compensation scheme is required for ICG imaging to allow for the clearance of agent (and hence loss of signal) from the bloodstream. Desmettre et al (cited above) note that ICG has only 4% of the fluorescence efficiency of fluorescein, and that the transport mechanism of ICG within retinal or choroidal endothelial cells, as well as ICG diffusion kinetics are poorly understood.

The design and mechanism of action of the contrast agents of the present invention, together with their pharmacokinetic properties, make them suitable for a number of in vivo imaging applications. Those properties include:
1) the agents have a prolonged retention in blood, with a whole body elimination half-life in small animals of approximately 8 hours. This property gives a stable and prolonged imaging signal for several hours. For vascular imaging applications, that means a stable signal from the vasculature is present, giving a prolonged imaging time window. A consequent advantage is that the agent can be given at a convenient time. Thus, an intravenous administration to the subject does not have to be given close to the imaging procedure, but can be given some time in advance.
2) the fluorescence signal is strong.
3) The spectral properties are sufficiently close to those of ICG to permit the use of existing detection systems and apparatus.
4) When the optical reporter fluoresces at wavelength over 700 nm, that is outside the visible spectral window (400-700 nm), hence a separate imaging channel can be provided for parallel fluorescent imaging that does not adversely influence the spectral properties of a white light imaging channel.
5) When the optical reporter fluoresces at wavelength over 600 nm, the reporter is relatively robust to photobleaching, hence can be imaged on multiple occasions without loss of signal.

### DETAILED DESCRIPTION OF THE INVENTION

In a first aspect, the present invention provides a method of in vivo optical imaging comprising:
(i) providing an optical imaging contrast agent suitable for in vivo imaging, said contrast agent comprising a conjugate of a synthetic polyethyleneglycol polymer of molecular weight 15 to 45 kDa, with one or two groups Opt^{R};
(ii) generating an optical image of a region of interest of a mammalian subject to which said contrast agent has been administered, said region of interest comprising of at least a portion of the blood vessels and/or blood pool of said subject;
wherein each Opt^{R} is independently a biocompatible optical reporter group capable of detection either directly or indirectly in an optical imaging procedure using light of wavelength 480-850 nm.

By the term “optical imaging” is meant any method that forms an image for detection, staging or diagnosis of disease, follow up of disease development or for follow up of disease treatment based on interaction with light in the green to near-infrared region (wavelength 500-1200 nm). Optical imaging further includes all methods from direct visualization without use of any device and involving use of devices such as various scopes, catheters and optical imaging equipment, eg. computer-assisted hardware for tomographic presentations. The modalities and measurement techniques include, but are not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarization, luminescence, fluorescence lifetime, quantum yield, and quenching. Further details of these techniques are provided by: (Tuan Vo-Dinh (editor): “Biomedical Photonics Handbook” (2003), CRC Press LCC; Mycek & Pogue (editors): “Handbook of Biomedical Fluorescence” (2003), Marcel Dekker, Inc.; Splinter & Hopper: “An Introduction to Biomedical Optics” (2007), CRC Press LCC.

By the term “optical imaging contrast agent” is meant a compound suitable for optical imaging of a region of interest of the whole (ie. intact) mammalian body in vivo. Preferably, the mammal is a living human subject. The imaging may be invasive (eg. intra-operative or endoscopic) or non-invasive.

The term “imaging at least a portion of the blood vessels” refers to imaging the inside or lumen of the blood vessel of the body, in particular the heart, arteries and veins. Such imaging is sometimes referred to by the general term ‘angiography’. Since blood vessels form part of organs and vessels within the mammalian body, imaging the blood vessels can give information on the function, perfusion and/or disease state of the organ/vessel/area of interest.

The term “blood pool” has its conventional meaning in the field of medical imaging, i.e. where the behaviour of the blood within the bloodstream of the subject can be imaged by virtue of the contrast agent circulating in the bloodstream. If for example, the contrast agent is present at a steady concentration in the bloodstream throughout the imaging study, then any changes observed can be attributed to other effects in the physiology or disease state of the mammalian subject. By imaging the wash-in phase of a bolus, information on perfusion can be obtained. By imaging the wash-out phase of a bolus, information on leakage rate can be obtained.

By the term “mammalian subject” is meant a living mammalian patient, preferably a living human subject.

The term “synthetic” has its conventional meaning, i.e. man-made as opposed to being isolated from natural sources. Such compounds have the advantage that their manufacture and impurity profile can be fully controlled.

The term “polyethyleneglycol polymer” or “PEG” has its conventional meaning, as described eg. in “The Merck Index”, 14^{th} Edition entry 7568, i.e. a liquid or solid polymer of general formula H(OCH₂CH₂)ₙOH where n is an integer greater than or equal to 4. The polyethyleneglycol polymers of the present invention may be linear or branched, but are preferably linear. The polymers are also preferably non-dendrimeric. The polyethyleneglycol polymer is suitably polydisperse. By the term “polymer terminus” is meant the functional group(s) which form the end of the polyether chains of the PEG polymer chains—in the above general formula the two hydroxy (—OH) groups.

By the term “conjugate” is meant a derivative in which the “optical reporter” (Opt^{R}) is covalently bonded to the polyethyleneglycol polymer.

By the term “biocompatible” is meant non-toxic and hence suitable for administration to the mammalian body, especially the human body, without adverse reaction, or pain or discomfort on administration.

By the term “optical reporter” (i.e. Opt^{R}) is meant a fluorescent dye or chromophore which is capable of detection either directly or indirectly in an optical imaging procedure using light of wavelength 480-850 nm. Since the optical reporter must be suitable for imaging the mammalian body in vivo, it must also be biocompatible. Preferably, the Opt^{R} has fluorescent properties, and it preferably comprises a fluorescent, biocompatible dye. When the Opt^{R} is suitable for use with light of wavelength 600-850 nm, that is a preferred wavelength region for intra-operative applications, when getting signal from depth within tissue is useful. When the Opt^{R} is suitable for use with light of wavelength 480-600 nm, that is a preferred wavelength region for surface angiography techniques such as retinal angiography. Suitable dyes for imaging in the 480-600 nm region are known in the art, and include fluorescein (excitation maximum 494 nm, emission maximum 520 nm and Cy3 (excitation maximum 546 nm, emission maximum 563 nm).

The term “region of interest” or ROI has its conventional meaning in the field of in vivo medical imaging.

### Preferred Features.

The molecular weight of polyethyleneglycol polymer is preferably 20-43 kDa, more preferably 22-40 kDa, and most preferably 25-38 kDa, with 27-35 kDa being the ideal. The polyethyleneglycol polymer is preferably a linear polymer.

The polyethyleneglycol polymer preferably only has conjugated thereto the Opt^{R} group(s). Thus, the polymer preferably does not have conjugated thereto a biological targeting molecule or other polymer. By the term “biological targeting moiety” is meant a compound which, after administration, is taken up selectively or localises at a particular site of the mammalian body.

The conjugate of the first aspect is preferably of Formula I:

Y¹—X^{a}-[POLYMER]-X^{b}—Y² (I)
where:
[POLYMER] is the synthetic polyethyleneglycol polymer;
X^{a} and X^{b} are attached at the termini of said polyethyleneglycol polymer, and are independently a bond or an L group;
   where L is a linker group of formula -(A)ₘ- wherein each A is independently —CR₂—, —CR═CR—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid, or a sugar; where each R is independently chosen from H, C₁₋₄ alkyl, C₂₋₄ alkenyl, C₂₋₄ alkynyl, C₁₋₄ alkoxyalkyl or C₁₋₄ hydroxyalkyl;
   m is an integer of value 1 to 20;
Y¹ and Y² are independently Opt^{R} or a functional group chosen from —OH; —O(C₁₋₁₀ alkyl); —NH₂ or —NH(CO)(C₁₋₁₀ alkyl);
   wherein Opt^{R} is as defined above;
with the proviso that at least one of Y¹ and Y² is Opt^{R}.

By the term “amino acid” is meant an L- or D-amino acid, amino acid analogue (eg. naphthylalanine) or amino acid mimetic which may be naturally occurring or of purely synthetic origin, and may be optically pure, i.e. a single enantiomer and hence chiral, or a mixture of enantiomers.

By the term “sugar” is meant a mono-, di- or tri-saccharide. Suitable sugars include: glucose, galactose, maltose, mannose, and lactose. Optionally, the sugar may be functionalised to permit facile coupling to amino acids. Thus, eg. a glucosamine

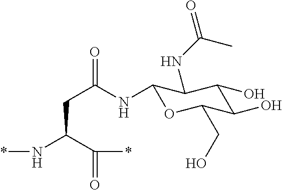 [SMILES: *N[C@@H](CC(=O)NC1OC(CO)C(O)C(O)C1NC(C)=O)C(*)=O]

derivative of an amino acid can be conjugated to other amino acids via peptide bonds. The glucosamine derivative of asparagine (commercially available from NovaBiochem) is one example of this:

In Formula I, when only one of Y¹ and Y² is Opt^{R}, the other is preferably a functional group chosen from —OH and —NH₂, more preferably —OH.

In Formula I, it is preferred that each of Y¹ and Y² is Opt^{R}. In that instance, X and X′ are preferably chosen to be —NHCO— or —CONH— such that the conjugate is prepared from a diamino-PEG or dicarboxy-PEG polymer. Such PEG polymers thus correspond to H₂N-[POLYMER]—NH₂ or HOOC-[POLYMER]-COOH respectively, wherein the biocompatible dye of Opt^{R} is conjugated to the polymer at each terminus via an amide bond.

When each of Y¹ and Y² is Opt^{R}, it is preferred that the Opt^{R} groups of Y¹ and Y² each comprise the same biocompatible reporter. That has three advantages. Firstly, when the two chromophores of the biocompatible reporters are the same, the contrast agent exhibits an enhanced fluorescent signal for effectively the same molecular weight (because the molecular weight of the reporter is so much less than that of the polymer). Secondly, possible unwanted interference and/or quenching of fluorescence between the signals from two different biocompatible reporters is avoided. Thirdly, symmetric bifunctional-PEGs are easier to synthesise than unsymmetrical ones.

In Formula I, m of the L group is preferably an integer of value 1 to 5, most preferably 1 to 3.

The Opt^{R} preferably comprises a biocompatible dye capable of detection either directly or indirectly in an optical imaging procedure using light of wavelength 600-850 nm, more preferably 610-800 nm, yet more preferably 700-780 nm, most preferably 730-770 nm. The biocompatible dye of Opt^{R} preferably has fluorescent properties. Particular examples of such dyes include: indocyanine green, the cyanine dyes Cy5, Cy5.5, Cy7, and Alexa Fluor 633, Alexa Fluor 647, Alexa Fluor 660, Alexa Fluor 680, Alexa Fluor 700, and Alexa Fluor 750.

The biocompatible dye is preferably a cyanine dye or benzopyrylium dye, most preferably a cyanine dye. Preferred cyanine dyes which are fluorophores are of Formula II:

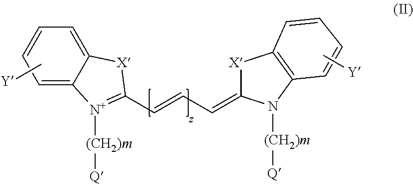

wherein:
each X′ is independently selected from: —C(CH₃)₂, —S—, —O— or
—C[(CH₂)ₐCH₃][(CH₂)_{b}M]-, wherein a is an integer of value 0 to 5, b is an integer of value 1 to 5, and M is group G or is selected from SO₃M¹ or H;
   each Y′ independently represents 1 to 4 groups selected from the group consisting of:
H, —CH₂NH₂, —SO₃M¹, —CH₂COOM¹, —NCS, F and a group G, and wherein the Y′ groups are placed in any of the positions of the aromatic ring;
   Q′ is independently selected from the group consisting of: H, SO₃M¹, NH₂, COOM¹,
ammonium, ester groups, benzyl and a group G;
   M¹ is H or B^{c}; where B^{c} is a biocompatible cation;
   z is an integer of value 2 or 3;
   and m is an integer from 1 to 5;
   wherein at least one of X′, Y′ and Q′ comprises a group G;
   G is a reactive or functional group suitable for attaching to the PEG polymer.

By the term “biocompatible cation” (B^{c}) is meant a positively charged counterion which forms a salt with an ionised, negatively charged group, where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred biocompatible cations are sodium and potassium, most preferably sodium.

The G group reacts with a complementary group of the PEG polymer forming a covalent linkage between the cyanine dye fluorophore and the polymer. The location of the G groups in Formula II is such that the PEG can suitably be conjugated at positions, Q′, X′ or Y′. G may be a reactive group that may react with a complementary functional group of the PEG, or alternatively may include a functional group that may react with a reactive group of the PEG. Examples of reactive and functional groups include: active esters; isothiocyanate; maleimide; haloacetamide; acid halide; hydrazide; vinylsulfone; dichlorotriazine; phosphoramidite; hydroxyl; amino; sulfydryl; carbonyl; carboxylic acid and thiophosphate. Preferably G is an active ester.

By the term “activated ester” or “active ester” is meant an ester derivative of the associated carboxylic acid which is designed to be a better leaving group, and hence permit more facile reaction with nucleophile, such as amines. Examples of suitable active esters are: N-hydroxysuccinimide (NHS), sulfo-succinimidyl ester, pentafluorophenol, pentafluorothiophenol, para-nitrophenol, hydroxybenzotriazole and PyBOP (i.e. benzotriazol-1-yl-oxytripyrrolidinophosphonium hexafluorophosphate). Preferred active esters are N-hydroxysuccinimide or pentafluorophenol esters, especially N-hydroxysuccinimide esters.

### Preferred Features of the Cyanine Dye.

Preferred cyanine dyes based on Formula II are as defined in Formula IIa:

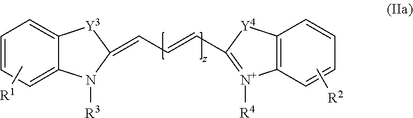

where:
   Y³ and Y⁴ are independently —O—, —S—, —NR⁵— or —CR⁶R⁷— and are chosen such that at least one of Y³ and Y⁴ is —CR⁶R⁷—;
   R¹ and R² are independently H, —SO₃M¹ or R^{a};
   R³ to R⁵ are independently C₁₋₅ alkyl, C₁₋₆ carboxyalkyl or R^{a};
   R⁶ is H or C₁₋₃ alkyl;
   R⁷ is R^{a} or C₁₋₆ carboxyalkyl;
   R^{a} is independently C₁₋₄ sulfoalkyl;
   where M¹ and z are as defined in Formula II;
with the proviso that the cyanine dye of Formula IIa comprises at least one R^{a} group and a total of 1 to 6 sulfonic acid substituents from the R¹, R² and R^{a} groups.

By the term “sulfonic acid substituent” is meant a substituent of formula —SO₃M¹, where M¹ is as defined above. Preferred dyes of Formula IIa have z=3. Preferred such dyes also have 2 to 6 sulfonic acid substituents. The —SO₃M¹ substituent is covalently bonded to a carbon atom, and the carbon atom may be aryl (such as the R¹ or R² groups), or alkyl (i.e. an R^{a} group). In Formula IIa, the R^{a} groups are preferably of formula —(CH₂)ₖSO₃M¹, where M¹ is as defined above, and k is an integer of value 1 to 4. k is preferably 3 or 4. Cyanine dyes which are more preferred in Formula IIa have z=3, i.e. are heptamethine cyanine dyes.

Particularly preferred cyanine dyes are of Formula IIb:

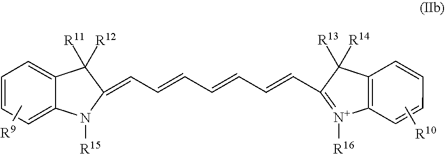

where:
   R⁹ and R¹⁰ are independently H or SO₃M¹, and at least one of R⁹ and R¹⁰ is SO₃M¹;
   R¹¹ and R¹² are independently C₁₋₄ alkyl or C₁₋₆ carboxyalkyl;
   R¹³, R¹⁴, and R¹⁶ are independently R^{b} groups;
   wherein R^{b} is C₁₋₄ alkyl, C₁₋₆ carboxyalkyl or —(CH₂)_{q}SO₃M¹,
      where q is an integer of value 3 or 4;
   where M¹ is as defined for Formulae II and IIa;
   with the proviso that the cyanine dye has a total of 1 to 4 SO₃M¹ substituents in the R⁹, R¹⁰ and R^{b} groups.

Preferred cyanine dyes of Formula IIb are chosen to comprise at least one C₁₋₆ carboxyalkyl group, or activated ester thereof, in order to facilitate conjugation to the PEG polymer. An especially preferred such dye of Formula IIb is Cy7:

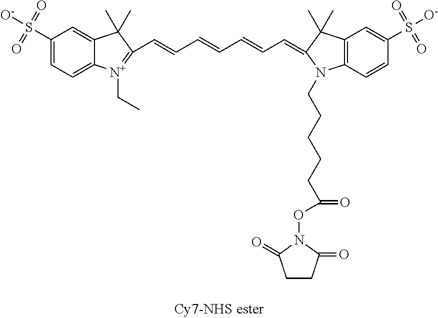 [SMILES: CC[N+]1=C(/C=C/C=C/C=C/C=C2\N(CCCCCC(=O)ON3C(=O)CCC3=O)c3ccc(S(=O)(=O)[O-])cc3C2(C)C)C(C)(C)c2cc(S(=O)(=O)[O-])ccc21]

The term “benzopyrylium dye” has its conventional meaning. Suitable benzopyrylium dyes of the present invention are denoted Bzp^{M} and are of Formula III:

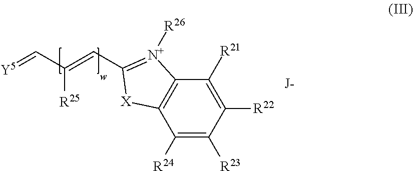

where:
   Y⁵ is a group of Formula Y^{a} or Y^{b}

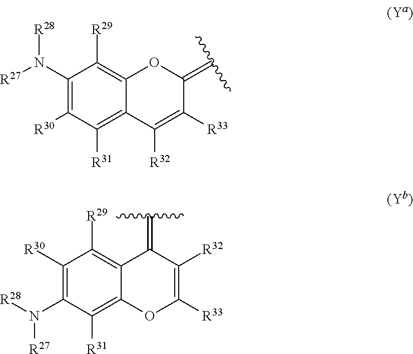

X is —CR³⁴R³⁵—, —O—, —S—, —Se—, —NR³⁶— or —CH═CH—, where R³⁴ to R³⁶ are independently R⁹ groups;
R²¹-R²⁴ and R²⁹-R³³ are independently selected from H, —SO₃M¹, Hal, R^{g} or C₃₋₁₂ aryl;
R²⁵ is H, C₁₋₄ alkyl, C₁₋₆ carboxyalkyl, C₃₋₁₂ arylsulfonyl, Cl, or R²⁵ together with one of R²⁶, R³⁴, R³⁵ or R³⁶ may optionally form a 5- or 6-membered unsaturated aliphatic, unsaturated heteroaliphatic or aromatic ring;
R²⁶ and R³⁶ are independently R^{g} groups;
R²⁷ and R²⁸ are independently C₁₋₄ alkyl, C₁₋₄ sulfoalkyl or C₁₋₆ hydroxyalkyl or for Y^{a} may optionally together with one or both of R²⁹ and/or R³⁰ may form a 5- or 6-membered N-containing heterocyclic or heteroaryl ring, or for Y^{b} may optionally together with one or both of R³⁰ and/or R³⁰ may form a 5- or 6-membered N-containing heterocyclic or heteroaryl ring;
R^{g} is C₁₋₄ alkyl, C₁₋₄ sulfoalkyl, C₁₋₆ carboxyalkyl or C₁₋₆ hydroxyalkyl;
w is 1 or 2;
J is a biocompatible anion;
where M¹ is as defined for Formula II;
with the proviso that Bzp^{M} comprises at least one sulfonic acid substituent chosen from the R²¹ to R³⁶ groups.

By the term “biocompatible anion” (J) is meant a negatively charged counterion which forms a salt with an ionised, positively charged group (in this case an indolinium group), where said negatively charged counterion is also non-toxic and hence suitable for administration to the mammalian body, especially the human body. The counterion (J⁻) represents an anion which is present in a molar equivalent amount, thus balancing the positive charge on the Bzp^{M} dye. The anion (J) is suitably singly- or multiply-charged, as long as a charge-balancing amount is present. The anion is suitably derived from an inorganic or organic acid. Examples of suitable anions include: halide ions such as chloride or bromide; sulfate; nitrate; citrate; acetate; phosphate and borate. A preferred such anion is chloride.

Suitable contrast agents of the invention are those wherein the Bzp^{M} is of Formula IIIa or IIIb:

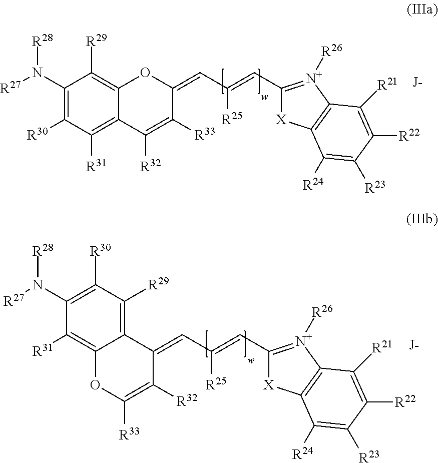

where X, w, J and R²¹-R³³ are as defined for Formula III.

When R²⁵ together with one of R²⁶/R³⁴-R³⁶ forms a 5- or 6-membered unsaturated aliphatic, unsaturated heteroaliphatic or aromatic ring, suitable such aromatic rings include: phenyl, furan, thiazole, pyridyl, pyrrole or pyrazole rings. Suitable unsaturated rings comprise at least the C═C to which R²⁵ is attached.

When R²⁷ and/or R²⁸ together with at least one of R²⁹, R³⁰ or R³¹ (depending on whether Y¹ is Y^{a} or Y^{b} as described above), form a 5- or 6-membered N-containing heterocyclic or heteroaryl ring, suitable such rings include: thiazole, pyridyl, pyrrole or pyrazole rings or partially hydrogenated versions thereof, preferably pyridyl or dihydropyridyl.

### Preferred Features of the Benzopyrylium Dye.

The PEG polymer is preferably attached at positions R²⁵, R²⁶, R³⁴, R³⁵ or R³⁶ of the Bzp^{M} of Formula III, more preferably R²⁶, R³⁴, or R³⁶ most preferably at R²⁶, R³⁴ or R³⁵. In order to facilitate the attachment the relevant R²⁵, R²⁶, R³⁴, R³⁵ or R³⁶ substituent preferably comprises C₁₋₆ carboxyalkyl, more preferably C₃₋₆ carboxyalkyl.

The benzopyrylium dye (Bzp^{M}) preferably has at least 2 sulfonic acid substituents, more preferably 2 to 6 sulfonic acid substituents, most preferably 2 to 4 sulfonic acid substituents. Preferably, at least one of the sulfonic acid substituents is a C₁₋₄ sulfoalkyl group. Such sulfoalkyl groups are preferably located at positions R²⁶, R²⁷, R²⁸, R³⁴, R³⁵ or R³⁶; more preferably at R²⁶, R²⁷, R²⁸, R³⁴ or R³⁵; most preferably at R²⁶ together with one or both of R²⁷ and R²⁸ of Formula III. The sulfoalkyl groups of Formula III, are preferably of formula —(CH₂)ₖSO₃M¹, where M¹ is H or B^{c}, k is an integer of value 1 to 4, and B^{c} is a biocompatible cation (as defined above). k is preferably 3 or 4.

In Formula III, w is preferably 2. R²⁵ is preferably H or C₁₋₄ carboxyalkyl, and is most preferably H. X is preferably —CR³⁴R³⁵— or —NR³⁶—, and is most preferably —CR³⁴R³⁵—. Especially preferred benzopyrylium dyes having w=2 are DY-750 and DY-752, which are commercially available from Dyomics GmbH.

In the method of the first aspect, the contrast agent preferably comprises a pharmaceutical composition of the conjugate, together with a biocompatible carrier. The “biocompatible carrier” is a fluid, especially a liquid, in which the imaging agent can be suspended or dissolved, such that the composition is physiologically tolerable, i.e. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is isotonic); an aqueous solution of one or more tonicity-adjusting substances (eg. salts of plasma cations with biocompatible counterions), sugars (e.g. glucose or sucrose), sugar alcohols (eg. sorbitol or mannitol), glycols (eg. glycerol), or other non-ionic polyol materials (eg. polyethyleneglycols, propylene glycols and the like). When a macromolecular polyol is used, it is suitably of molecular weight up no more than 10 kDa, preferably below 5 kDa—since higher molecular weight species might compete with the contrast agent of the present invention. Preferably, the biocompatible carrier is pyrogen-free water for injection or isotonic saline.

The pharmaceutical compositions may be prepared under aseptic manufacture (i.e. clean room) conditions to give the desired sterile, non-pyrogenic product. It is preferred that the key components, especially the associated reagents plus those parts of the apparatus which come into contact with the imaging agent (eg. vials) are sterile. The components and reagents can be sterilised by methods known in the art, including: sterile filtration, terminal sterilisation using e.g. gamma-irradiation, autoclaving, dry heat or chemical treatment (e.g. with ethylene oxide). It is preferred to sterilise some components in advance, so that the minimum number of manipulations needs to be carried out. As a precaution, however, it is preferred to include at least a sterile filtration step as the final step in the preparation of the pharmaceutical composition. The pharmaceutical compositions are preferably prepared from a kit, as is described in the fourth aspect (below).

The method of the first aspect is particularly suitable for vascular imaging, which is here used to refer to the heart and vessels for conveying blood (eg. veins or arteries) making up the peripheral vasculature.

Optical imaging of the vasculature of gastric tumours is preferably carried out endoscopically. Imaging gastric tumours within a short time of administration of the imaging agent is preferred. Rapid imaging post-administration is feasible with the agents of the present invention, since they behave as blood pool agents, i.e. exhibit a prolonged half-life in blood. Consequently, a single injection can be used to produce vascular signal for an extended period, allowing a single dose to be used for imaging the entire stomach. ICG is used for this application, but the rapid blood clearance complicates the imaging protocol.

The prolonged vascular phase of the agents of the present invention is also advantageous for imaging applications in ophthalmology, such as age related macular degeneration (AMD). Thus, imaging at a relatively early time (hours post-administration (can be compared to late phase imaging (days post-administration), which will reveal the presence of any extravascular compound, and this would indicate leakage from angiogenic vessels, a feature of AMD.

The imaging method of the first aspect may preferably be carried out using tomographic imaging. That technique is particularly useful for imaging eg. breast cancer. Such imaging has been carried out successfully in humans with a fluorescence optical tomography system using ICG. ICG enables detection of tumours due to their increased vasculature and total blood content. ICG is used in this application as a blood pool agent. One of the disadvantages of using ICG is its short blood half-life, and the fact that the tomography data acquisition takes several minutes to perform. Accordingly, a compensation scheme is required for ICG imaging. The agents of the present invention exhibit very stable kinetics in blood compared to the time of the scan, which obviates the need for compensation schemes. Consequently, repeat scanning at early times (a few hours post administration), can be compared to scans in the late phase (24 hours plus). The early time images will indicate blood pool imaging, and the late phase will be more specific to the agent that has leaked from the vasculature and accumulated locally.

The prolonged, stable blood concentration of the present imaging agents has similar advantages when imaging rheumatoid arthritis, for the reasons described above.

The region of interest (ROI) of the first aspect is preferably a tumour, the eye, a blood vessel or an arthritic joint. For tumour imaging, the method is particularly suitable for imaging tumours in body cavities, including but not limited to stomach, colon/rectum, cervix, bladder, oral cavity/oesophagus, and bronchi. The method is also particularly suited for imaging tumours in organs which are amenable to tomographic imaging, such as the breast or the prostate. Finally, the method is suitable for imaging of blood vessels during surgical resection of tumours. For imaging blood vessels it is particularly suitable for imaging function and patency of blood vessel grafts such as coronary artery bypass procedures. For the imaging of arthritis, the joint is preferably a finger, foot, elbow, wrist or knee joint, more preferably a finger joint (since fingers are typically affected first).

When the region of interest is the eye, the method is particularly suitable for imaging a mammalian subject suffering from age-related macular degeneration (AMD).

The method of the first aspect may also be used to determine the kinetics of leaking, i.e. how quickly or slowly the agent leaks out of leaky vasculature. Thus, in angiography for example, areas of leaky vasculature in the retina can be visualised when following a bolus injection of agent over a few minutes. The rate at which the agent leaks out of the blood vessels (producing a diffuse signal in adjacent tissue) can be observed. This could be useful for, e.g. retinal angiography and/or applications such as imaging vasculature of stomach lesions endoscopically.

Multiple administration of the agent may also lead to clinically useful information. Thus, the contrast agent is first administered (dose #1) to the subject say 18-24 hours before imaging. A second bolus administration is given during imaging (dose #2), then information on both vasculature and the leakage component may be gathered at the same time. Thus, a low imaging signal will be present from dose #1 agent that has leaked out of the vasculature. Subtracting that signal from the dose #2 signal, will reveal only the bolus signal. This could allow perfusion (from bolus kinetics) and vascular delineation and leakage information to be obtained rapidly—since if the dose #1 was not given, it may take too long to see leakage adequately in a short imaging examination procedure.

A preferred optical imaging method of the first aspect is Fluorescence Reflectance Imaging (FM). In FRI, the contrast agent of the present invention is administered to a subject to be diagnosed, and subsequently a tissue surface of the subject is illuminated with an excitation light—usually continuous wave (CW) excitation. The light excites the Opt^{R} of the contrast agent. Fluorescence from the contrast agent, which is generated by the excitation light, is detected using a fluorescence detector. The returning light is preferably filtered to separate out the fluorescence component (solely or partially). An image is formed from the fluorescent light. Usually minimal processing is performed (no processor to compute optical parameters such as lifetime, quantum yield etc.) and the image maps the fluorescence intensity. The contrast agent is designed to concentrate in the disease area, producing higher fluorescence intensity. Thus the diseased area produces positive contrast in a fluorescence intensity image. The image is preferably obtained using a CCD camera or chip, such that real-time imaging is possible.

The wavelength for excitation varies depending on the particular dye used. The apparatus for generating the excitation light may be a conventional excitation light source such as: a laser (e.g., ion laser, dye laser or semiconductor laser); an array of LEDs; halogen light source or xenon light source. Various optical filters may optionally be used to obtain the optimal excitation wavelength.

In a first embodiment, a preferred FRI method comprises the steps of:
(i) a tissue surface comprising the region of interest within the animate subject is illuminated with an excitation light;
(ii) fluorescence from the contrast agent, which is generated by excitation of the Opt^{R} is detected using a fluorescence detector;
(iii) the light detected by the fluorescence detector is optionally filtered to separate out the fluorescence component;
(iv) an image of said tissue surface is formed from the fluorescent light of steps (ii) or (iii).

In the method comprising steps (i)-(iv), the excitation light of step (i) is preferably continuous wave (CW) in nature.

In a second embodiment, the optical imaging preferably comprises FDPM (frequency-domain photon migration). This has advantages over continuous-wave (CW) methods where greater depth of detection of the dye within tissue is important [Sevick-Muraca et al, Curr. Opin. Chem. Biol., 6, 642-650 (2002)]. For such frequency/time domain imaging, it is advantageous if the Opt^{R} has fluorescent properties which can be modulated depending on the tissue depth of the lesion to be imaged, and the type of instrumentation employed. A preferred FDPM method comprises the steps of:
(a) exposing light-scattering biologic tissue having a heterogeneous composition, said tissue forming a region of interest of said animate subject, to light from a light source with a pre-determined time varying intensity to excite the contrast agent, the tissue multiply-scattering the excitation light;
(b) detecting a multiply-scattered light emission from the tissue in response to said exposing;
(c) quantifying a fluorescence characteristic throughout the tissue from the emission by establishing a number of values with a processor, the values each corresponding to a level of the fluorescence characteristic at a different position within the tissue, the level of the fluorescence characteristic varying with heterogeneous composition of the tissue; and
(d) generating an image of the tissue by mapping the heterogeneous composition of the tissue in accordance with the values of step (c).

The fluorescence characteristic of step (c) preferably corresponds to uptake of the contrast agent and preferably further comprises mapping a number of quantities corresponding to adsorption and scattering coefficients of the tissue before administration of said contrast agent. The fluorescence characteristic of step (c) preferably corresponds to at least one of fluorescence lifetime, fluorescence quantum efficiency, fluorescence yield and contrast agent uptake. The fluorescence characteristic is preferably independent of the intensity of the emission and independent of contrast agent concentration.

The quantifying of step (c) preferably comprises: (i) establishing an estimate of the values, (ii) determining a calculated emission as a function of the estimate, (iii) comparing the calculated emission to the emission of said detecting to determine an error, (iv) providing a modified estimate of the fluorescence characteristic as a function of the error. The quantifying preferably comprises determining the values from a mathematical relationship modelling multiple light-scattering behaviour of the tissue. The method of the first option preferably further comprises monitoring a metabolic property of the tissue in vivo by detecting variation of said fluorescence characteristic.

The contrast agents of the first aspect can be prepared as follows:

In order to facilitate conjugation of the Opt^{R} to the PEG polymer, the dye of the Opt^{R} suitably has attached thereto a reactive functional group (Q^{a}). The Q^{a} group is designed to react with a complementary functional group of the polymer, thus forming a covalent linkage between the dye and the polymer. Suitable Q^{a} groups may be selected from: carboxyl; activated esters; isothiocyanate; maleimide; haloacetamide; hydrazide; vinylsulfone, dichlorotriazine and phosphoramidite. Preferably, Q^{a} is: an activated ester of a carboxylic acid; an isothiocyanate; a maleimide; or a haloacetamide. Most preferably Q^{a} is an activated ester. Preferred aspects of such activated esters are as described above.

General methods for conjugation of cyanine dyes to biological molecules are described by Licha et al [Topics Curr. Chem., 222, 1-29 (2002); Adv. Drug Deliv. Rev., 57, 1087-1108 (2005)]. Methods for conjugating cyanine dyes to PEG polymers are taught by Licha et al [SPIE Vol 3196 p. 98-102 (1998)].

When the conjugate comprises two Opt^{R} groups, one at each terminus of the PEG polymer, a preferred starting material is a diamino-PEG. As noted by Elbert et al, [Elbert & Hubbell; Biomacromol., 2, 430-441 (2001)], such diamino-PEG materials can be of low purity. For the conjugates of the present invention, the PEG-diamine is preferably of greater than 90% purity, more preferably of over 95% purity, most preferably of over 99% purity. The synthesis described by Elbert provides PEG-diamines of the required purity. Example 1 provides further details.

Cyanine dyes functionalised suitable for conjugation to peptides are commercially available from GE Healthcare Limited, Atto-Tec, Dyomics, Molecular Probes and others. Most such dyes are available as NHS esters. Methods of conjugating the linker group (L) to the polymer employ analogous chemistry to that of the dyes alone (see above), and are known in the art. Benzopyrylium dyes are commercially available from Dyomics GmbH, Winzerlaer Str. 2A, D-07745 Jena, Germany; (www.dyomics.com).

In a second aspect, the present invention provides a method of monitoring the effect of treatment of a mammalian subject with a drug, which comprises the method of imaging of the first aspect, where the imaging is effected before and after treatment with said drug, and optionally also during treatment with said drug.

Preferred aspects of the imaging method and of the contrast agent in the second aspect are as described in the first aspect (above).

In a third aspect, the present invention provides a method of diagnosis of the mammalian body, which comprises the method of imaging of the first aspect.

Preferred aspects of the imaging method and of the contrast agent in the third aspect are as described in the first aspect (above).

In a fourth aspect, the present invention provides the use of the optical imaging contrast agent as defined in the first aspect in either:
(i) the method of imaging of the first aspect;
(ii) the method of monitoring of the second aspect;
(iii) the method of diagnosis of the third aspect.

Preferred aspects of the contrast agent in the fourth aspect are as described in the first aspect (above).

The contrast agent of the fourth aspect is preferably prepared using a kit, hence this aspect includes such a use, where the contrast agent is prepared from a kit. Also included, is the use of a kit comprising the contrast agents of the invention in the use of the fourth aspect.

The “kit” comprises the contrast agent as defined in the first aspect in sterile, solid form such that, upon reconstitution with a sterile supply of a biocompatible carrier (as described in the third aspect), dissolution occurs to give the desired pharmaceutical composition.

In that instance, the contrast agent, plus other optional excipients as described above, may be provided as a lyophilised powder in a suitable vial or container. The agent is then designed to be reconstituted with the desired biocompatible carrier to give the pharmaceutical composition in a sterile, apyrogenic form which is ready for mammalian administration. A preferred sterile, solid form of the contrast agent is a lyophilised solid. The sterile, solid form is preferably supplied in a pharmaceutical grade container, as described for the pharmaceutical composition (above). When the kit is lyophilised, the formulation may optionally comprise a cryoprotectant chosen from a saccharide, preferably mannitol, maltose or tricine.

### DESCRIPTION OF THE FIGURES

FIG. 1 shows the blood clearance vs time of Compound 1 in rats.

FIG. 2 shows a fluorescence image taken 1 hour after iv injection of Compound 1 in the rat MatBIII tumour model.

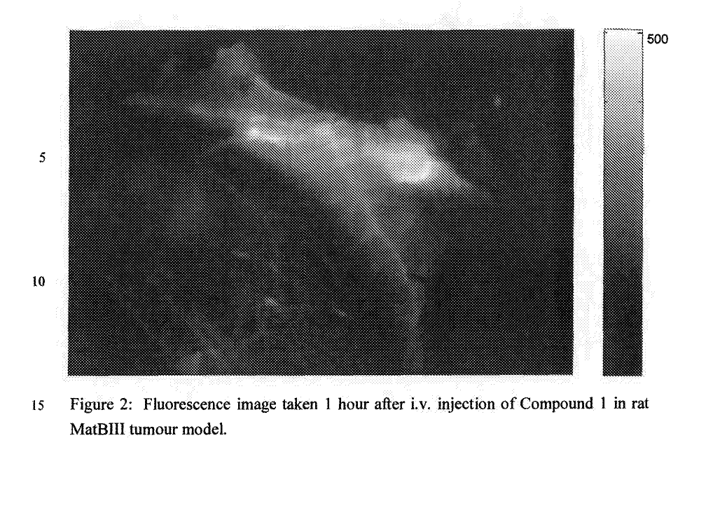

The invention is illustrated by the non-limiting Examples detailed below. Example 1 provides the synthesis of a PEG-bis(dye) conjugate of the invention. Example 2 provides the synthesis of other PEG-dye conjugates of the invention. Example 3 demonstrates the blood clearance of Compound 1 of the invention in vivo. Example 4 describes in vivo imaging using Compound 1. A representative image is shown in FIG. 2. Blood vessels and blood-rich tumour can be clearly seen, i.e. show positive image contrast.

### ABBREVIATIONS

Conventional 3-letter and single letter amino acid abbreviations are used.
Acm: Acetamidomethyl
ACN: Acetonitrile
ADME: adsorption, distribution, metabolism and excretion.
Boc: tert-Butyloxycarbonyl
DMF: N,N″-Dimethylformamide
DMSO: Dimethylsulfoxide
GFC: gel filtration chromatography
HCl: Hydrochloric acid
HPLC: High performance liquid chromatography
ID: injected dose.
MALDI: Matrix assisted laser desorption ionization.
NHS: N-hydroxy-succinimide.
PBS: Phosphate-buffered saline.
TFA: Trifluoroacetic acid.

### EXAMPLE 1

### Synthesis of a Bis-Cy7 PEG-31k Conjugate (Compound 1)

Diamino-PEG was purchased from supplier LaysanBio. It was synthesised from the corresponding PEG-diol (Sigma/Aldrich), using the method of Elbert et al [Biomacromolecules, 2, p 430-441 (2001)]. The diamine-PEG had an average mass of ˜31 kDa by GFC and ˜35 kDa by MALDI. Amine substitution was ca. 100% with no other impurities detectable by proton NMR, in particular no CH₂—OMs or CH₂—OH protons observable.

The fluorescent dye, Cy7-NHS was obtained from GE Healthcare. It had an active ester content of 81.3%. The conjugate was prepared as follows:

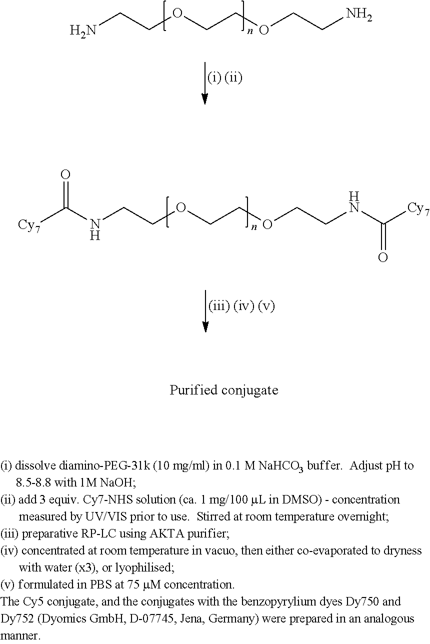

### EXAMPLE 2

### Synthesis of Other PEG-Dye Conjugates

PEGs functionalised with a single dye molecule were synthesised in an analogous manner to Example 1, using the appropriate PEG-monoamine with the dye active ester (˜1.2-1.5 equivalents).

The PEG 43 kDa conjugate was prepared by reaction of mono-amino PEG20K with a bifunctional dye (Cy5-bis NHS ester) in a molar ratio of 3.33:1. Thus, PEG20K (100 mg) was co-evaporated with anhydrous DMF (3×) and redissolved in anhydrous DMF (5 ml). To this solution N-methylmorpholine (4 μl) was added followed by a solution of Cy5-bis NHS (0.3 equiv. in 146 μl of DMSO). The mixture was stirred in the dark overnight and then purified by HPLC. The pure fraction was concentrated using an Amicon 5K MWCO filter.

### EXAMPLE 3

### Blood Clearance of Compound 1 In Vivo

In vivo studies were carried out with female Fischer 344 rats or severe combined immunodeficient (SCID) mice, each with an age between 4 and 8 weeks. Animals were housed with non-fluorescent food (Harlan Labs, cat. #TD.97184) and water ad libitum and a standard 12 hour day-night lighting cycle. 1×10⁶ cells (100 μL) were injected, using a 27 gauge needle, directly orthotopically into the mammary fat pad of the animal. After allowing 7 days for MatBIII tumor growth (˜1 cm in diameter), animals were injected with test agent and imaged.

The ADME profile of Compound 1 was studied by performing optical biodistribution studies in the MatB III in vivo orthotopic tumor model. Thus, a dose of 125 nmol/kg (volume ˜250-300 μL) was used for the study with an equal volume of PBS used as a negative control. For naïve animals, each tissue type was split into four tubes to ensure sufficient volume for standard preparation. Tissues included blood, bladder/urine, liver, kidney, spleen, eyes, fat, muscle, tumor, and skin collected from the contralateral side from tumor. Using the supernatant from naïve samples (pooled by tissue type), standards were made by adding agent at concentrations of 75 μM, 37.5 μM, 3.75 μM, 0.375 μM and 0 μM, dilutions made with PBS. This yielded a final concentration of 20% injected dose (ID), 10%, 1%, 1% and 0. To determine the amount of dye contained in each tissue type, 90 μL of sample or standard was read on a spectrophotometer at settings of 710 excitation/805 Emission. From this data, fluorescence was expressed as % ID/g of tissue.

Samples were adjusted for weight, but there was some sample-to-sample variation.

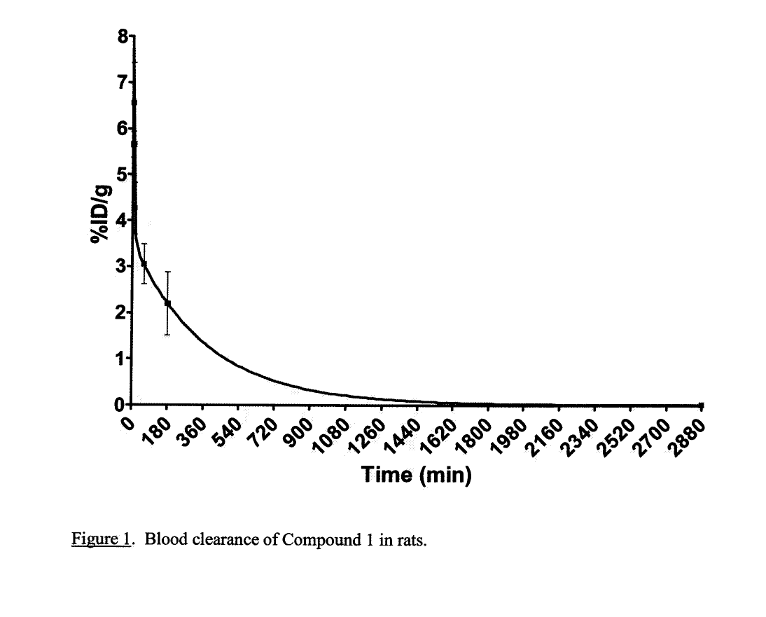

The clearance profile of the agent is shown in Table 1. The blood clearance is shown in FIG. 1. A bi-phasic exponential decay curve fit was used to determine the half-life of the agent with a t_{1/2α}=4.6 min and t_{1/2β}=260 min. Most of the organs related to metabolism and excretion (liver, kidney, spleen) showed little uptake and accumulation with less than 2% ID/g in every organ. At 48 h post injection, little retention of the agent was seen in all of the collected tissues with <1% ID/g. In conclusion, little non-specific organ accumulation was observed.

TABLE 1
Compiled biodistribution data of Compound 1 in MatBIII
tumor-bearing rats.

| Time | | | | Kid- | | | | |
|---|---|---|---|---|---|---|---|---|
| (min) | Blood | Bladder | Liver | ney | Spleen | Skin | Eye | Tumor |
| 2 | 6.56 | 0.00 | 0.24 | 0.12 | 0.64 | 0.00 | 1.93 | 0.00 |
| 5 | 5.65 | 0.23 | 0.46 | 1.35 | 0.64 | 0.56 | 0.14 | 0.53 |
| 10 | 4.26 | 1.24 | 0.03 | 0.41 | 1.00 | 0.00 | 1.70 | 0.00 |
| 60 | 3.05 | 0.86 | 1.02 | 0.51 | 0.28 | 0.05 | 0.33 | 0.00 |
| 180 | 2.19 | 0.00 | 2.04 | 0.63 | 0.32 | 0.06 | 0.12 | 0.05 |
| 2880 | 0.02 | 0.00 | 0.23 | 0.00 | 0.00 | 0.00 | 0.44 | 0.61 |

### EXAMPLE 4

### Blood Clearance of Compound 1 In Vivo

Compound 1 was studied in the MatBIII model described in Example 3. The dose was either 125 or 250 nmol dye/kg body weight. The fluorescence imaging system was comprised of a 12 bit cooled CCD camera (Hamamatsu ORCA), and with a 735 laser source providing the excitation light. The fluorescence signal was filtered with a band pass emission filter set (810 nm centre frequency, 90 nm bandwidth). 50 ms exposure time was used with 2×2 binning.

For immobilisation during the optical imaging procedure, the animals were anaesthetized in a coaxial open mask to surgical level anaesthesia with Isoflurane (typically 2-3%) with oxygen as the carrier gas. During injection and intermediate time point imaging a lighter anaesthesia level was used. The animals were imaged from above, lying on their backs. The animals were supplied external heating from a heating blanket to sustain normal body temperature for the duration of the imaging. Each animal was given one contrast agent injection in the tail vein. The injection volume was dependent on the concentration of the test substance, ranging from 0.2 to 0.4 ml. Digital images were acquired and stored continuously from right before and the first two minutes after contrast injection using a Hamamatsu ORCA ER high sensitivity digital camera and Wasabi Imaging Software (Hamamatsu, Germany).

FIG. 2 shows an image 1-hour post-injection of Compound 1.

What is claimed is:

1. A method of in vivo optical imaging comprising:
(i) providing an optical imaging contrast agent suitable for in vivo imaging, said contrast agent comprising a conjugate of a synthetic polyethyleneglycol polymer of molecular weight 15 to 45 kDa, with one or two groups Opt^{R};
(ii) generating an optical image of a region of interest of a mammalian subject to which said contrast agent has been administered, said region of interest comprising of at least a portion of the blood vessels and/or blood pool of said subject;
wherein each Opt^{R} is independently a biocompatible optical reporter group capable of detection either directly or indirectly in an optical imaging procedure using light of wavelength 480-850 nm.

2. The method of claim 1, where the polymer has conjugated thereto only the Opt^{R} group(s).

3. The method of claim 1, where the conjugate is of Formula I:
Y¹—X^{a}-[POLYMER]-X^{b}—Y² (I)
where:
[POLYMER] is the synthetic polyethyleneglycol polymer;
X^{a} and X^{b} are attached at the termini of said polyethyleneglycol polymer, and are independently a bond or an L group;
where L is a linker group of formula -(A)ₘ- wherein each A is independently —CR₂—, —CR═CR—, —C≡C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid, or a sugar;
where each R is independently chosen from H, C₁₋₄ alkyl, C₂₋₄ alkenyl, C₂₋₄ alkynyl, C₁₋₄ alkoxyalkyl or C₁₋₄ hydroxyalkyl;
m is an integer of value 1 to 20;
Y¹ and Y² are independently Opt^{R} or a functional group chosen from —OH; —O(C₁₋₁₀ alkyl); —NH₂ or —NH(CO)(C₁₋₁₀ alkyl);
with the proviso that at least one of Y¹ and Y² is Opt^{R}.

4. The method of claim 3, where each of Y¹ and Y² is Opt^{R}.

5. The method of claim 4, where the Opt^{R} groups of Y¹ and Y² each comprise the same biocompatible optical reporter.

6. The method of claim 1, where the biocompatible optical reporter is a cyanine dye.

7. The method of claim 1, where the biocompatible optical reporter group is a benzopyrylium dye.

8. The method of claim 1, where the polyethyleneglycol polymer has a molecular weight of 22 to 40 kDa.

9. The method of claim 1, where the polyethyleneglycol polymer is a linear polymer.

10. The method of claim 1, where the contrast agent comprises a pharmaceutical composition of the conjugate, together with a biocompatible carrier.

11. The method of claim 1, where the optical imaging comprises tomographic imaging.

12. The method of claim 1, where the region of interest within said subject is a tumour, the eye or an arthritic joint.

13. The method of claim 12, where the tumour is gastric, stomach or breast cancer.

14. The method of claim 12, where the eye is imaged and the mammalian subject is suffering from age-related macular degeneration (AMD).

15. A method of monitoring the effect of treatment of a mammalian subject with a drug, which comprises the method of imaging of claim 1, where the imaging is effected before and after treatment with said drug, and optionally also during treatment with said drug.

16. A method of diagnosis of the mammalian body, which comprises the method of imaging of claim 1.

17. (canceled)

18. (canceled)
